(19) United States
(12) Patent Application Publication
(10) Pub. No.: US 2015/0066071 A1
(43) Pub. Date: Mar. 5, 2015

(54) NASAL DILATOR AND BOUGIE

(71) Applicant: SIMPLICITY, LLC, Springville, UT (US)

(72) Inventors: Ian J. Alexander, Los Alamos, NM (US); Brian D. Owens, Plano, TX (US)

(21) Appl. No.: 14/477,655

(22) Filed: Sep. 4, 2014

Related U.S. Application Data
(60) Provisional application No. 61/873,706, filed on Sep. 4, 2013.

(51) Int. Cl. A61F 5/08; A61M 29/00

(52) U.S. Cl. CPC A61F 5/08; A61M 29/00; A61F 2250/001; A61M 2210/0618; A61M 2210/0681
USPC 606/199

(57) ABSTRACT

A nasal dilator and bougie is disclosed. The dilator includes, for example, an elongated member terminating in an end and a slidable member having a terminal end movable to the end of the elongated member in a first position. A dilation portion at the end of the elongated member has a first diameter and a second diameter greater than the first diameter when the slidable member is in the first position. An actuator can move the slidable member.

### RELATED APPLICATIONS

This Application claims priority to pending U.S. provisional patent application Ser. No. 61/873,706 entitled “Nasal Dilator”, filed Sep. 4, 2013, the entire contents of which are hereby incorporated by reference in their entirety.

This Application is related to co-pending U.S. utility patent application Ser. No. 13/654,401 entitled “Systems and Methods for Controlling Balloon Catheters”, filed Oct. 17, 2012 and U.S. utility patent application Ser. No. 13/654,409 entitled “Endoscopic Peripheral” filed Oct. 17, 2012 the entire contents of which are all hereby incorporated by reference in their entirety.

### BACKGROUND

Each year more and more dilation procedures, such as sinus dilation, are performed. For example, balloon sinuplasty, brain surgery, enteroscopy, rhinoscopy, esophagogastroduodenoscopy, and cosmetic surgery may involve dilation through the nose or other orifice (natural or surgically created) of a patient out of necessity or convenience. In particular, balloon sinuplasty has become more popular in recent years because of enhanced equipment and minimal down time for the patient. Balloon sinuplasty is an endoscopic surgical procedure for the treatment of conditions, such as blocked nasal sinuses. Because the procedure involves the insertion into the nose of balloon catheters, guide wires, and other devices and instruments, such as irrigation catheters, illumination systems, and navigation systems, the costs associated with the required systems may be quite expensive. In addition, the costs are high due to the FDA approval process, manufacturing, and packing limitations and expenses associated with those processes and likewise insurance reimbursements for the devices, equipment, staff, and hours have decreased placing significant pressure on medical professionals and medical service providers.

Properly performing dilation procedures and surgeries in the body of the patient require correct positioning of the necessary instrumentation, devices, and equipment. Some procedures may require multiple medical professionals to ensure proper guidance and placement of the equipment due the size and awkwardness of the equipment. As a result of the complex nature of the systems and procedures and expense of the equipment, some medical professionals may feel uncomfortable performing balloon sinuplasty or similar procedures.

### SUMMARY

One embodiment provides a nasal dilator. The nasal dilator can include an articulable body including a shaft with a first end. The nasal dilator may also include a bougie slidably disposed about the shaft and having an actuated position adjacent the dilation tip.

In another embodiment, a nasal dilator is disclosed. The dilator includes, for example, an elongated member terminating in an end and a slidable member having a terminal end movable to the end of the elongated member in a first position. A dilation portion at the end of the elongated member has a first diameter and a second diameter greater than the first diameter when the slidable member is in the first position.

Still another embodiment provides a nasal dilator with bougie. The dilator can include a shaft terminating in a tip and a slidable bougie having a dilation end movable from a retracted position to at least the tip of the shaft in a dilation position. The nasal dilator has a first diameter at the tip in the retracted position and a second diameter at the tip greater than the first diameter in the dilation position.

Yet another embodiment provides a sinus dilator method. An elongated member terminating in an end and a slidable member having a terminal end is provided. The slidable member is movable to a first position at the end of the elongated member. By moving the slidable member to the first position, a dilation portion at the end of the elongated member is changed from a first diameter to a second diameter greater than the first diameter.

### BRIEF DESCRIPTION OF THE DRAWINGS

Illustrative embodiments of the present invention are described in detail below with reference to the attached drawing figures, which are incorporated by reference herein and wherein:

FIG. 1A is a schematic, plan view of a portion of a sinus dilator in accordance with an illustrative embodiment;

FIG. 1B is a schematic, plan view of a portion of another sinus dilator in accordance with an illustrative embodiment;

FIG. 1C is a schematic, plan view of a portion of another sinus dilator in accordance with an illustrative embodiment;

FIG. 1D is a schematic, hidden view of a portion of a sinus dilator system in accordance with an illustrative embodiment;

FIGS. 2A-2E are schematic, plan views of dilation heads of sinus dilators in accordance with an illustrative embodiment;

FIG. 3 is a schematic, partial cut-away view of a bougie dilation system in accordance with an illustrative embodiment;

FIG. 4 is a schematic, partial cut-away view of another sinus dilation system in accordance with an illustrative embodiment;

FIG. 5 is a schematic, exploded view of the sinus dilation system of FIG. 4 in accordance with an illustrative embodiment;

FIG. 6 is a schematic, partial cut-away view of another sinus dilation system in accordance with an illustrative embodiment;

FIG. 7 is a schematic, partial cut-away view of another sinus dilation system in accordance with an illustrative embodiment;

FIG. 8 is a schematic, partial cut-away view of another sinus dilation system in accordance with an illustrative embodiment;

FIG. 9 is a schematic, partial cut-away view of another sinus dilation system in accordance with an illustrative embodiment;

FIG. 10 is a schematic, partial cut-away view of another sinus dilation system in accordance with an illustrative embodiment;

FIG. 11 is a schematic, partial cut-away view of another sinus dilation system in accordance with an illustrative embodiment;

FIG. 12 is a flowchart of a process for utilizing a sinus dilator in accordance with an illustrative embodiment

FIGS. 13A-13D are schematic, cut-away views of a bougie being utilized for sinus dilation in accordance with an illustrative embodiment;

FIG. 14 is a schematic, partial cut-away of a another dilation system in accordance with an illustrative embodiment;

FIG. 15 is a schematic, plan view of a sinus dilator in accordance with an illustrative embodiment;

FIG. 16 is a schematic, plan view of a sinus dilator in accordance with an illustrative embodiment; and

FIG. 17 is a flowchart of a process for utilizing a sinus dilator in accordance with an illustrative embodiment.

### DETAILED DESCRIPTION OF THE DRAWINGS

Illustrative embodiments provide a dilation system, method, and dilation devices for performing dilation. In one embodiment, the dilators represent a dilator along with a bougie is guided or slid to perform nasal dilation. The various systems, devices, and tools with their numerous features and configurations are referred to herein as a “dilator” or “dilators.” In one embodiment, the dilators may be utilized for sinus dilation of the frontal, maxillary, sphenoid, and ethmoid sinuses and are referred to as nasal dilators. In other embodiments, the dilators may also be utilized for other dilation procedures, such as nasal opening, choannal atresia, rectal narrowing, or dilation in any number of orifices, organs, tissues, muscles, arteries, or surgically created openings. References to sinus dilation procedures are synonymous with the different types of internal or external medical procedures that may be performed utilizing the various embodiments.

The dilators may be rigid, semi-rigid, or flexible (e.g., bendable, malleable, etc.). For example, the sinus dilator may be bent by hand or utilizing a tool to customize the shape, configuration, relative angle of one portion to another, and entry angle of the dilation tip(s) of the sinus dilator.

Likewise, the dilators may be configured for one-time usage or may be utilized repeatedly. For example, the dilators may be configured to be cleaned utilizing any number of sterilization and cleaning devices, systems, or processes. In one embodiment, the dilators are formed from medical or surgical grade metals, plastic, polymers, or other similar materials. For example, surgical metals utilized to form all or portions of the dilators may include stainless steel (e.g., austenitic 316, martensitic 440 and 420), titanium alloys (e.g., Ti6A14V), Tecason, Tecapeek, Tecanyl, Tecatron, Tecapro, Tecaform, Tecanat, Tecafine, Tecadur, Tecatec, Tecapei, and other like plastics or having similar material properties. The dilators may also be configured to have antimicrobial, antibacterial, clotting, or other properties or to disperse one or more medications or materials throughout the surface or defined openings of the dilators as needed.

In one embodiment, the dilators may include a dilation head or tip configured to enter and fit one or more of the sinuses. For example, the dilation heads may include a rounded, angled, or tapered shape for performing dilation of the sinus or orifice without damaging tissues or otherwise injuring or causing excessive pain to the patient. The dilation heads may also have any number of other shapes and configurations suitable for dilation.

In one embodiment, the diameter of the dilation heads may vary between 1 mm-6 mm or more or less as needed with the 2 mm-5 mm being sized for most human patients. Dilation of the sinuses to between 2 mm to 5 mm is desirable for most patients. The dilators may also be specially configured and sized for children (e.g., infants, toddlers, adolescents, etc.), adults, animals, and other orifices or applications. In one embodiment, the dilators may have a dilation tip with different angles, slopes, and shapes that are configured to impinge upon the sinus pathways to perform dilation. For example, the medical professional may manipulate the dilation systems and devices to position the tip of the dilation system within the sinus as well as advance the tip into the sinus pathways to perform the dilation.

In one embodiment, the dilators may be semi-bendable for shaping the dilator for the specific geometry and positioning of the sinuses in the nose of a user/patient. For example, the medical professional may bend, mold and/or shape the dilator before using it on the user/patient. The dilator may be bent by hand or utilizing a tool to achieve a desired angle or configuration. In another embodiment, all or portions of the nasal dilator may be three dimensionally printed.

In another embodiment, the dilator may include an attachable or removable head. The dilation head (which may be extended to include a neck and portion of a handle) is the portion of the dilator that may be inserted within the user to perform dilation. The removable head may allow a medical professional to maintain sterility. For example, a disposable head retrieved from a sterile package may be connected to a reusable handle for performing the dilation procedure after which the disposable head is disposed of. In addition, by disposing of only the removable head, costs and waste may be decreased significantly and potential insurance reimbursements for the dilator may be kept at reasonable amounts. In other embodiments, the entire dilator may be disposable.

The dilator may include connectors at ends of a shaft or handle for connecting a first and second dilation head and a first end and second end of the dilator, respectively. In another embodiment, a first connector may be utilized to connect a first dilation tip with a lumen at the first end and suction, irrigation, camera, or light through the second connector at the second end.

The dilator may also include one or more external rounded grooves, channels, ducts (internal or external), or dimples for allowing fluids space to flow, potentially reducing suction within the body of the user, communicating medicine (e.g. dip the dilator in a medication before performing the procedure or employ a material configured to excrete a like medicinal constituent), or enhancing the ease with which the dilator may be introduced into the sinus. For example, the outer surface of the dilation tip that enters the body of the patient that may be dipped in medication before the dilation procedure is performed to apply the medication to the affected areas during dilation. Other possibilities for performing like functions include using, for example, one or more geometries, materials or combination of both to control interaction of the boundary surface (e.g., surface tension) with the surrounding tissue (e.g., surface tension parameters to promote or discourage fluid wicking, fluid flow and surface repulsion (hydrophobicity).

In one embodiment, the dilators include markings or indicators that act as reference points for the medical professional. The markings may be lines, text, or symbols that indicate the approximate depth that the dilation system or device should be inserted into the nose or body of the user. The markings may be configured to be incandescent or glow in the dark to increase visibility to the user in a number of conditions, environments, and light conditions. For example, during sinus procedures, the markings may prevent a medical professional from reaching the brain of the patient with the dilator.

In one embodiment, the dilator may include integrated or removable dual heads (ends) with each of the heads configured for separate sinuses or dilation procedures. The dual heads may further increase the flexibility and ease of use of the dilators for the medical professionals. For example, the dual heads may be of different sizes (e.g. 2 mm and 4 mm, or 3 mm and 5 mm) to perform dilation in stages to more effectively perform dilation without injuring the patient. The dilators herein described may utilize any number of handles or grips or mechanisms for moving and manipulating the dilator to best help the user during the dilation procedure.

In one embodiment, the dilator may include one or more internal channels, lumens, or tubes for applying suction or irrigation. For example, the dilator may include a hollow core for performing suction or irrigation (or both) during a dilation procedure. In addition to suction, and possibly separately, the one or more channels may be configured with a surface property (e.g. resulting from geometric or material properties) to wick or draw fluids in one or more directions. Being able to perform suction during the procedure may be useful to prevent unwanted mucus, blood, and other build up during the procedure. The channels (e.g. internal, as well as external) may also be utilized to receive a fiber optic camera, wireless camera, or light source for utilization at the end of the dilator. The hollow core may also be utilized to deliver irrigation, medication, or so forth to one or more exit ports or through all or portions of the surface area of the dilators. The hollow core dilators may also be flexible allowing for irrigation, suction, and/or medical instruments (e.g., fiber optic cameras, balloon catheters, injection devices, etc.).

In another embodiment, the nasal dilator may have a C-shape or a slit along all or a portion of the nasal dilator. The lumen may then be used to inflate an elongated balloon within the nasal dilator to perform additional dilation. Inflation of the balloon opens the nasal dilator increasing the diameter to perform dilation.

In another embodiment, the dilator may be utilized as part of a dilation system to extend a bougie that performs dilation. The dilator may act as a guide for a bougie that is integrated with, attached to, or mounted on the dilator. The bougie may be advanced based on user feedback (e.g., the user manually pushing or pulling the bougie or components attached to the bougie), mechanical advancement, pneumatic/hydraulic advancement, or electrical advancement.

In one embodiment, the dilators may be stand-alone devices that are easily manipulated. In another embodiment, the dilators may be integrated with other systems, such as optical systems (e.g., fiber optic or wireless cameras), suction systems, medication delivery systems, or so forth.

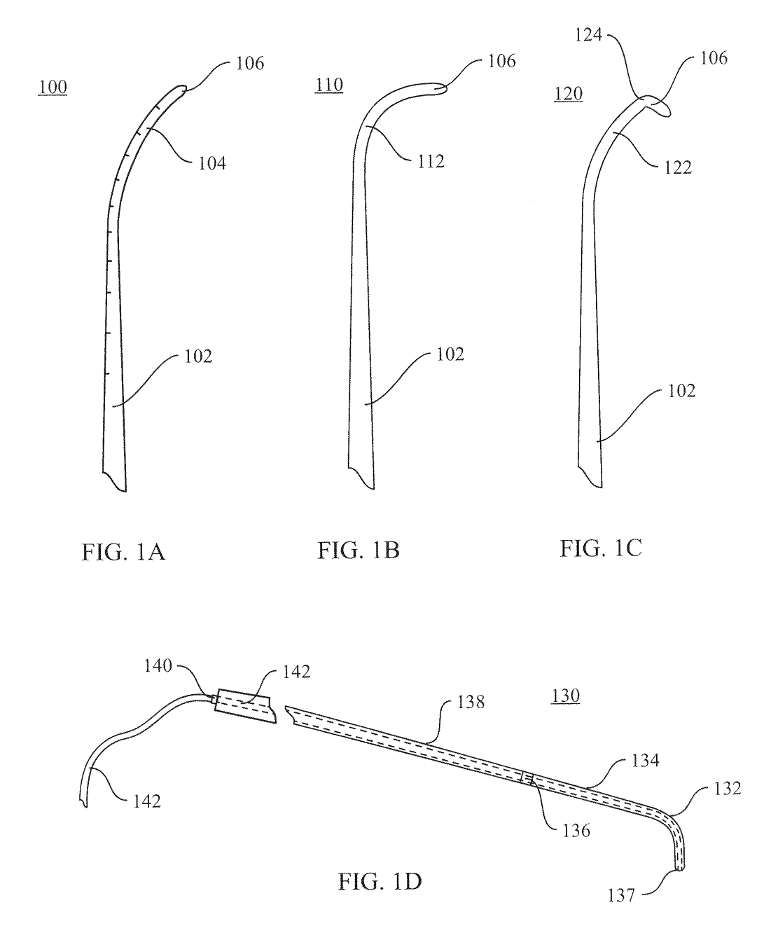

FIG. 1A is a schematic, plan view of a portion of a sinus dilator 100 in accordance with an illustrative embodiment. In one embodiment, the sinus dilator 100 may include a shaft 102 a neck 104, and the dilation tip 106. For example, the sinus dilator 100 may be configured to perform dilation of the sphenoid sinus. The shaft 102, neck 104, and dilation tip 106 may be formed from a single piece of material, such as metal, plastic, polymers, or combinations of each. In another embodiment, a head portion of the sinus dilator 100 may be configured to be attached to or removed from the shaft 102.

Although not shown, the shaft 102 may be configured to extend further for manipulation by the patient; extension of the shaft 102 may perform telescopically or by expansion of a flexible material. In another embodiment, the shaft 102 may include a handle, grips, or other like components for manipulating the sinus to 100. For example, the shaft 102 may be threaded into a handle or may include a coupling mechanism or snap attachment for attachment to a grip, handle or other like manipulatable feature. In other embodiments, the sinus dilator 100 may be configured to be utilized with other medical devices, systems, or equipment.

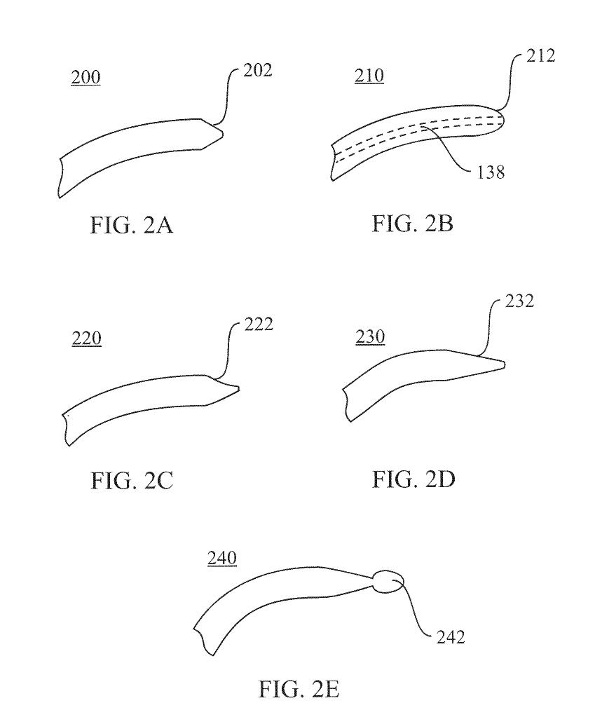

The neck 104 of the sinus dilator 100 may be configured and manipulated to reach the sinus being evaluated and dilated. The dilation tip 106 is the portion of the sinus dilator 100 that impinges upon the sinus walls to perform dilation. The angle and shape of the dilation tip 106 may vary as are further shown in FIGS. 2A-2E.

As shown, the sinus dilator 100 may have a slight curve for accessing and dilating the frontal sinuses. In one embodiment, the sinus dilator 100 may include an inner core or structural skeleton/frame (e.g. rigid or formable) (not shown) for retaining a shape, such as metal or plastic, with an outer plastic skin, coated rubber, or polymer surface that is low friction (also not shown). The metal core, frame or skeleton may allow the sinus dilator 100 to be bent or formed into position allowing the sinus dilator 100 to be customized for each patient. The outer surface provides reduced friction for easy insertion into the nose of the patient. Both the skeleton and the skin may be configured of a material that is hand-bendable. The core, frame or skeleton of the sinus dilator 100 may be of a material retains its bent shape thereby maintaining the outer coating/skin in the same bent shape as the core, frame or skeleton. The frame or skeleton may be configured as a laminate type material where one of the layers of the laminate is capable of retaining its hand-bent shape. The core, frame or skeleton may be formed from a hand-bendable metal, plastic, rubber, or composite that maintains its shape for accessing and dilating the sinuses. The sinus dilator 100 may have a body formed from a single material, such as a shape memory material or a hand-bendable plastic, rubber, metal or like material.

Any number of materials, such as metal, plastic, polymers, or composites may be utilized to configure the shape, and curvature of the shaft 102, the neck 104, and the dilation tip 106 for properly performing the dilation procedure.

Turning now to FIG. 1B illustrating a sinus dilator 110 in accordance with an illustrative embodiment. The sinus dilator 110 may include a different curvature of the neck 112 for accessing the maxillary sinus. The curvature of the neck 112 may be significant enough to access the maxillary sinus while still allowing the working portions of the sinus dilator 110 (those that are inserted within the patient) to be inserted and removed from the patient during the dilation procedure.

Turning now to FIG. 1C illustrating a sinus dilator 120 in accordance with an illustrative embodiment. The sinus dilator 120 may be configured for accessing and dilating the frontal sinuses. In addition to a neck 122, the sinus dilator 120 may include a curvature 124. The curvature 124 may represent an additional bend or corner utilized to access the frontal sinus. As previously mentioned for any of the embodiments of FIG. 1A, FIG. 1B, and FIG. 1C, the sinus dilator 120 may be completely rigid or fixed, or may be bendable or malleable based on a sufficient user-applied force to customize the shape, configuration, relative angle of one portion to another portion and entry angle of the sinus dilator 120.

Turning now to FIG. 1D illustrating a sinus dilation system 130 in accordance with an illustrative embodiment. In one embodiment, the sinus dilation system 130 may include a neck 132, a removable head 134, a release 136, a dilation tip 137, a channel 138, a port 140, and a hose 142. As previously described, the sinus dilation system 130 may include the removable head 134 which may include the neck 132, the dilation tip 137 and the release 136. The removable head 134 may correspond to the portion of the sinus dilation system 130 that may be inserted within the patient during the dilation procedure.

In other dilation procedures, the size and shape of the removable head 134 may vary to accommodate more of the sinus dilation system 130 being inserted by the medical professional during the dilation procedure. The removable head 134 may connect to the shaft 102 utilizing any number of known techniques or systems. Arresting the removable head 134 to the shaft 102 may be accomplished, for example, using mechanical, pneumatic (e.g. vacuum seat), magnetic, or other like means. For example, the shaft 102 may include a threaded portion corresponding to a threaded portion of the removable head 134 for rotationally attaching the removable head 134 to the shaft 102. The shaft 102 may also include a port that is configured to receive an adaptor and the removable head 134 for an interference fit based on the sizing and shape of the port and adaptor, such as a coupling system or couplers that provide sufficient forces to secure the two portions of the sinus dilation system 130. The shaft 102 may be configured with a seating portion for sealing against a like seating portion on the removable head 134 to vacuum couple the two portions together. The release 136 could be configured as a pressure release (e.g. actuated valve) to break the vacuum coupling and permit separation of the two portions. Similarly, the shaft 102 and removable head 136 could be configured with one or more magnets for releasably securing the two portions together.

In another embodiment, the release 136 may be a button, slide, or lever that must be activated by a medical professional in order to separate the shaft 102 from the removable head 134. For example, the removable head 134 may include a collared neck that fits within the shaft 102 and the release 136 may include a bearing mechanism that is utilized to secure the collar in position and release it when done. The release 136 may also be configured with a catch, dog, or spring-operated ball that secures the shaft 102 from the removable head 134. The removable head 134 and the other components and features of FIG. 1D may be equally applicable to the other described embodiments.

The sinus dilation system 130 may include one or more channels including the channel 138. The one or more channels may be utilized to apply suction, irrigation, or insert a fiber optic camera or wireless camera, and/or light source. The one or more channels may also be configured to house one or more elements, constructs, components, or means for manipulating the neck 112, dilation tip 106, curvature 124, or like portions/features of the sinus dilation system 130. In one embodiment, the channel 138 is between 0.2 mm to 0.8 mm in diameter. However, the channel 138 may be smaller or larger based on the size of the sinus dilation system 130 and based on the applicable dilation procedure. The channel 138 may also be configured to taper or narrow based on the corresponding size of the shaft 102, the neck 132, and the dilation tip 137. For example, the channel 138 may decrease in size as it approaches the dilation tip 137 to ensure that the portions of the channel 138 and the removable head 134 do not compromise the integrity and strength of the removable head 134.

In one embodiment, the shaft 102 may include a port 140 at a second end for receiving one or more attachments for suction, irrigation, cameras, medication delivery and/or light sources. For example, the hose 142 may represent a suction device, irrigation delivery device, fiber optic camera extension, light source, or other medical component. In another embodiment, the port 140 may be positioned at a mid-point of the dilation system 130 and shaft 102. The port 140 may extend perpendicularly from the shaft 102 or at an acute or obtuse angle. Other angles or positioning may also be utilized as necessary for the selected dilation procedure. The port 140 may provide an additional gripping point for the user/medical professional. The length of the port 140 may be increased (e.g. 1-4 inches) to provide another substantial hand hold to the user/medical professional.

Referring now to FIGS. 1A-1D, the sinus dilator 100 may be configured for dilation of the sphenoid sinuses, the sinus dilator 110 may be configured for the maxillary sinuses, and the sinus dilator 120 may be configured for the frontal sinuses. The sinus dilation system 130 may be bent for dilation procedures involving either the sphenoid or maxillary sinuses based on the requirements of the user/medical professional. The sinus dilators 100, 110 and 120, as well as the sinus dilation system 130 may include markings from the dilation tip extending all the way down along the shaft. For example, the sinus dilators 100, 110 and 120 and the sinus dilation system 130 may have millimeter and centimeter markings as well as numeric labels or symbols that help the user/medical professional determine how far into the patient's body the applicable instrument has, should be, or is inserted. The markings may also be bioluminescent or glow-in-the-dark, enabling the medical professional to make such determinations even in zero or low light conditions, or within the body of the patient.

Turning now to FIGS. 2A-2E showing various embodiments of dilation in 200, 210, 220, 230, and 240 in accordance with illustrative embodiments. The dilation ends 200, 210, 220, 230, and 240 may also be referred to as dilation heads. The dilation ends 200, 210, 220, 230, and 240 may utilize any number of shapes and configurations for a dilation tip including conical, frustoconical, rounded, spherical, oval shaped, flattened, concave, convex, joined, or so forth.

In one embodiment, the dilation tip is configured to avoid the uncinated bone during utilization. The dilation end 200 may include a dilation tip 202 that utilizes a rounded conical shape for insertion into the sinuses of a patient. The dilation tip 202 may include a flattened portion (e.g. shaped frustoconically) at the very tip or may go to a rounded point. The dilation tip may include one or more chamfered surfaces. The dilation end 210 may include a rounded dilation tip 212. The rounded dilation tip 212 is configured to prevent unwanted trauma to the sinuses of the patient. The dilation end 210 may also include the channel 138 utilized to house one or more tool operative components, a fiber optic camera, and/or light source, or apply suction/irrigation.

In one embodiment, the dilation end 210 may include miniature stops or limiters at the intersection of the dilation tip 212 and the channel 138 that prevent a fiber optic camera or light source from extending beyond the end of the dilation tip 212. However, in other embodiments, the dilation end 210 and the dilation tip 212 may be configured to allow the extension of a fiber optic camera, light source, irrigation system, or suction device beyond the dilation tip 212. The dilation end 220 may include a dilation tip 222 that may include a concave surface for performing dilation.

In other embodiments, distinct sides or edges of the dilation tip 222 may be concave, convex, straight, rounded, fluted, or so forth. The dilation end 230 may include a dilation tip 232. The dilation tip 232 may have a decreased entry angle for more gradually performing the dilation procedure. For example, the dilation tip 232 may vary between 1 mm in diameter at the very end of the dilation tip 232 to a diameter of 4 mm after a length of between 3 to 6 millimeters of the dilation tip 232.

In another embodiment, the dilation end 240 may include a dilation tip 242. As shown, the dilation tip 242 may include a ball, spherical or rounded shape at the end of the dilation tip 242 and a narrowing section before the ball dilation tip 242. The dilation tip 242 may be configured to help the medical professional find the sinus before the rest of the dilation end 200 is inserted into the sinus or other orifice for performing the dilation procedure. In one embodiment, the diameter of the ball, cone, or flattened shape may vary between 1-4 mm. The rest of the dilation end 240 may have a diameter of between 3-6 mm with large and smaller sizes contemplated based on the dilation procedure.

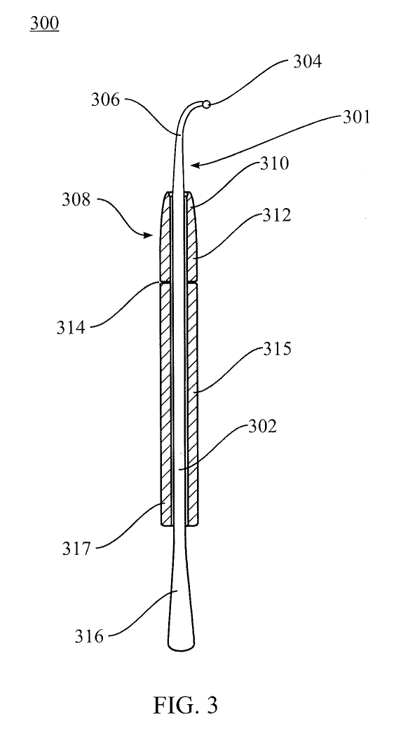

Turning now to FIG. 3 illustrating a bougie dilation system in accordance with an illustrative embodiment. The bougie dilation system 300 may also include any number of components, features, and characteristics including, but not limited to, a sinus dilator 301, a shaft 302, a dilation tip 304, a neck 306, and a slidable bougie 308. The slidable bougie 308 may further include a bougie tip 310, a bougie head 312, an attachment point 314, and a bougie body 315.

In another embodiment, the shaft 302 may extend to form a handle 316. In one embodiment, the bougie dilation system 300 may be utilized to perform an in-office dilation of one or more sinuses. The sinus dilator 301 may be inserted into the patient's nose and the dilation tip 304 may be inserted into the sinus of the patient. Next, the medical professional advances the slidable bougie 308 along the length of the sinus dilator 301 into the sinus to perform dilation of the selected sinus in which the dilation tip 304 is positioned. The slidable bougie 308 has sufficient material strength to dilate the sinuses while still being advanced along the sinus dilator 301. For example, the slidable bougie 308 may have longitudinal strength in the Y-direction for being advanced along the sinus dilator 301 without bunching or otherwise deforming significantly along the pathway. The slidable bougie 308 is flexible enough to bend in the X-direction when moving around the neck 306 to the dilation tip 304.

In one embodiment, the slidable bougie 308 is configured to move only to the end point of the dilation tip 304. For example, any number of stops, catches, or other like mechanical or structural configurations of the bougie tip 310 may stop the slidable bougie 308 at the dilation tip 304. However, in other embodiments, the slidable bougie 308 may be configured to extend beyond the end of the dilation tip 304 and the sinus dilator 301. The sinus dilator 301 may be formed of a single piece of material, such as metal or plastic. In other embodiments, one or more of a removable head or the handle 316 may be attached to the shaft 302 or other portions of the sinus dilator 301. As previously described, the sinus dilator 301 may be semi-malleable and shapeable, include one or more channels, or be otherwise configured for specialized applications.

In one embodiment, the sinus dilator 301 may be a sinus seeker manufactured by companies such as Entellus, Invotec, Millennium Surgical Corp., JEDMED, Bausch+Lomb Instruments, and BR Surgical. The slidable bougie 308 may be an integrated portion of the sinus dilator 301. For example, the slidable bougie 308 may be slidably attached to the sinus dilator 301 utilizing rails, channels, protrusions, guides, tracks, corrugations, or so forth for sliding along the length of the sinus dilator 301 and particularly the neck 306 and the dilation tip 304.

In another embodiment, the slidable bougie 308 may be inserted over the sinus dilator 301. For example, the sinus bougie 308 may be slid over the dilation tip 304 starting with an advancement end 317 of the bougie body 315 and finishing with the bougie tip 310.

In another embodiment, the handle 316 may be removed from the sinus dilator 301. As a result, the handle 316 may be removed and the slidable bougie 308 may be threaded/slid over the shaft 302 with the bougie tip 310 first to align the bougie dilation system 300 as is shown in FIG. 3.

As is described in the subsequent embodiments, the slidable bougie 308 may be a single component, or maybe two or more components that are attached, integrated, or otherwise connected to form a single component. In one embodiment, only the bougie head 312 is configured or designed to enter the body of the patient. As a result, the bougie body 315 may be utilized any number of times with only the bougie head 312 being replaced for different procedures. For example, the bougie head 312 may be 7-8 cm. long corresponding to the maximum depth that a dilation device should enter the patient's nasal pathways to perform dilation before approaching the patient's brain. The bougie head 312 may be composed of one or more layers, with the outer layer(s) having a greater formability and forgiveness than the inner layer(s). The relative greater rigidity of the outer layer(s) to the inner layer(s) allows the bougie head 312 to be advanced toward the dilation tip 304 without getting bound-up. The greater formability and forgiveness of the outer layer(s) also helps prevent causing inadvertent trauma to the sinuses. The greater rigidity of the inner layer(s) may also be used suitable connection point to removably attach the bougie head 312 to the bougie body 315.

In one embodiment, the bougie head 312 may be threadably attached to the bougie body 315. In another embodiment, the bougie head 312 may include a port and the bougie body 315 may include a connector for connecting the distinct components for utilization. The angle and configuration of the bougie tip 310 may vary as was previously described for the dilation tips between rounded, spherical, flattened, oval shaped, conical, frustoconical, and so forth to maximize the dilation procedure.

In one embodiment, a medical professional may utilize fingers, thumbs or other portions of the hand or body to advance the slidable bougie 318 once the dilation tip 304 is in place to perform the dilation procedure. For example, the user/medical professional may grip the handle 316 and utilize a thumb or finger(s) to advance the slidable bougie 308 toward the dilation tip 304.

In another embodiment, the bougie dilation system 300 may include a second end at the end of the handle 316 for performing dilation from either end. The slidable bougie 308 may be similarly two-sided and may be configured to be slid in either direction to perform dilation. For example, the slidable bougie 308 may be tapered at both ends of smoothly entering a sinus. The shaft 302 may also include a dilation tip as is described herein for dilating any number of openings, such as a sphenoid, maxillary, or frontal sinus.

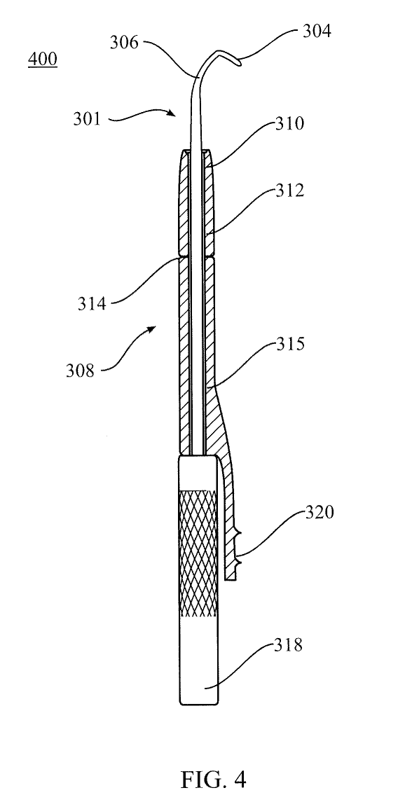

Turning now to FIG. 4, illustrating a bougie dilation system 400 in accordance with an illustrative embodiment. The bougie dilation system 400 may be configured for any of the sinuses as previously described. A thumb advancement 320 may extend from the bougie body 315. In one embodiment, the thumb advancement 320 is configured to allow the medical professional to utilize a thumb or finger(s) to advance the slidable bougie 308 along the sinus dilator 301. The thumb advancement 320 may include ridges, protrusions, corrugations, undulations, ribs, flutes, or other components that prevent the medical professional's thumb from slipping off of the thumb advancement 320 when advancing and retracting the slidable bougie 308. The thumb advancement 320 may also be configured of a highly tactile material that is easily gripped. The length of the thumb advancement 320 may be associated with the distance the slideable bougie 308 needs to enter the body of the user. As shown, the size and shape of the handle 318 may vary. For example, the handle 318 may be cylindrically shaped. The handle may also be ergonomically shaped to fit the medical professional's hand and fingers, including grooves, corrugations, undulations, ribs, flutes, ridges, grips, or so forth. In one embodiment, the handle 318 may include knurling to provide additional friction between the user's hand and the handle 318.

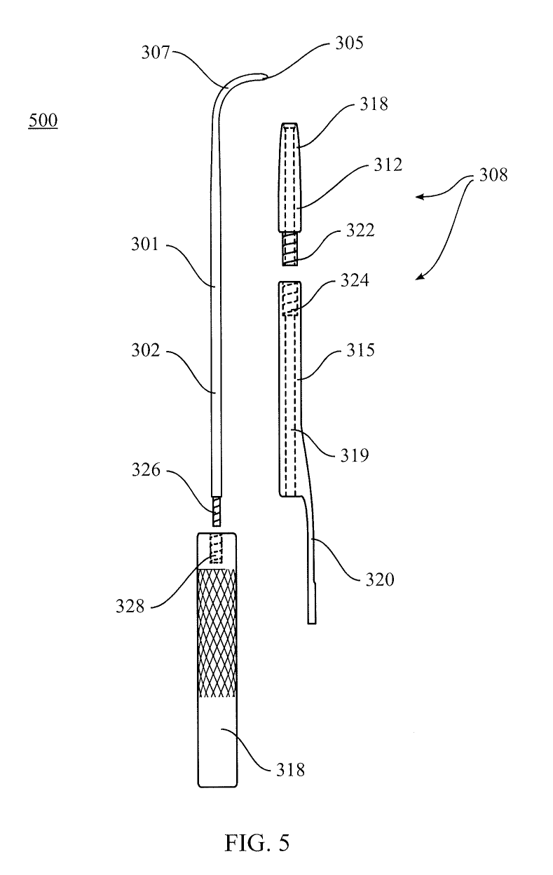

Turning now to FIG. 5 illustrating the separate components of a bougie dilation system 500 in accordance with an illustrative embodiment. In addition to those components previously described, the slidable bougie 308 may include a dilator channel 319 configured to receive the sinus dilator 301, as well as threads 322 and a port 324 configured to connect the bougie head 312 to the bougie body 315. For example, the threads 322 may be configured as male threading and as part of a connector of the bougie head 312 that may be threaded into the port 324 (e.g. female threaded port) of the bougie body 315. The sinus dilator 301 may also include threads 326 and a port 328 for connecting the shaft 302 to the handle 318. The respective fittings for connecting the bougie head 312 and bougie body 315, the shaft 302 and handle 308 may be configured with a coupling mechanism such as, ring coupling, compression coupling, or other like coupling. Other couplings such as a keyway, keyseat or keyed coupling may be used to create solid and rigid connection between the respective connected portions.

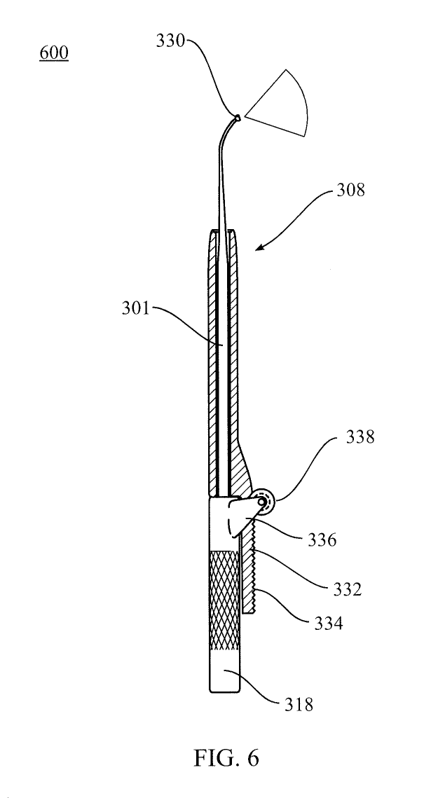

Turning now to FIG. 6, illustrating a bougie dilation system 600 in accordance with an illustrative embodiment. The bougie dilation system 600 may be similar to the other embodiments previously described. However, the slidable bougie 308 may be formed of a single piece of material instead of separate components. The slidable bougie 308 may also include an advancement section 332 defining teeth 334 (e.g. a rack with cogs). Likewise, the sinus dilator 301, or the handle 318 may include one or more supports 336 and a drive wheel 338 (e.g., a pinion) rotationally attached to the supports 336. For example, the drive wheel 338 may be rotationally attached to the supports 336 by an axle. In one embodiment, the supports 336 may be integrated with the handle 318. In another embodiment, the supports 336 may be configured to attach to the handle 318 utilizing a clamp, tabs, screws, tie, snaps, tether, or other mechanical attachments or fasteners. The drive wheel 338 may be smooth or may include teeth corresponding to the teeth 334 of the advancement section 332 (e.g. similar to a rack and pinion configuration). The drive wheel 338 may be driven by the medical professional's fingers or thumb to advance the slidable bougie 308 along the length of the sinus dilator 301. As shown, the sinus dilator 301 may also include a fiber optic camera 330 that is inserted within or integrated within the sinus dilator 301.

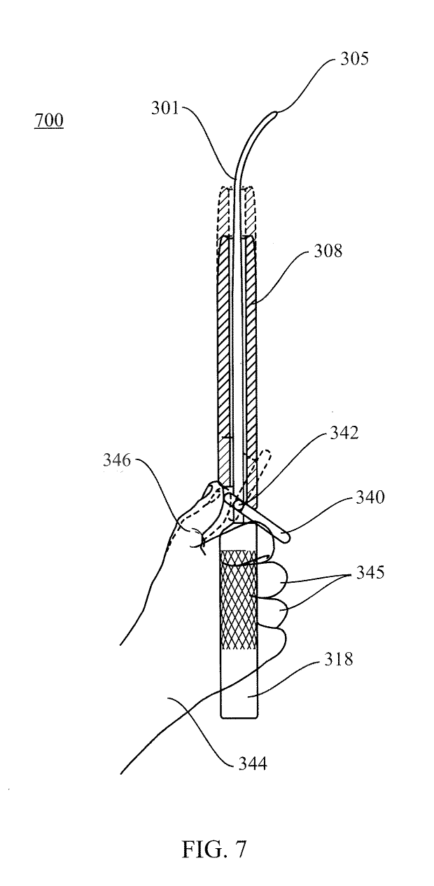

In another embodiment, the fiber optic camera 330 may be wireless and the wireless transceiver and other logical components may be integrated within the handle 318. FIG. 7 is a schematic, partial cut-away view of another sinus dilation system 700 in accordance with an illustrative embodiment. The sinus dilation system 700 may be configured to be operated utilizing a medical professional's hand 344, fingers 345 and thumb 346. In one embodiment, the sinus dilation system 700 may include a driver 340 and a pivot 342.

The driver 340 may drive the slidable bougie 308 through the designated path. In one embodiment, the driver 340 is a single component that impinges upon the slidable bougie 308 (e.g. rotation of driver 340 about pivot 342 engages slidable bougie 308). The driver 340 may also include multiple components, such as a linkage or driver arm connected to the driver (not shown) for driving a bottom edge of the slidable bougie 308. For example, the driver 340 may be configured as a pawl to engage a counterpoising feature on the slidable bougie 308. The driver 340 may contact the slidable bougie 308 on a single side, or around the outer periphery of the sinus dilator 301. In one embodiment, the driver 340 may be physically connected to the slidable bougie 308. For example, a hook, loop, latch, tab, catch, port, or other structure of the slidable bougie 308 may connect to or be integrated with the driver 340. In another embodiment, the driver 340 only impinges on the slidable bougie 308 during operation by the medical professional. The driver 340 may be configured having one or more varying geometries to provide a mechanical advantage to the medical professional for advancing the slidable bougie 308. For example, an end of the driver 340 may be configured with a thumb tab (not shown) to provide a mechanical advantage or leverage to the medical professional. The driver 340 may also be connected at the pivot point 342 closer to the end configured to engage the slidable bougie 308 to increase operational leverage on the slidable bougie 308.

In one embodiment, the driver 340 may be moved between positions utilizing the thumb of the medical professional. For example, by pulling the thumb 346 towards the handle 318, the slidable bougie 308 may be driven towards the dilation tip 305.

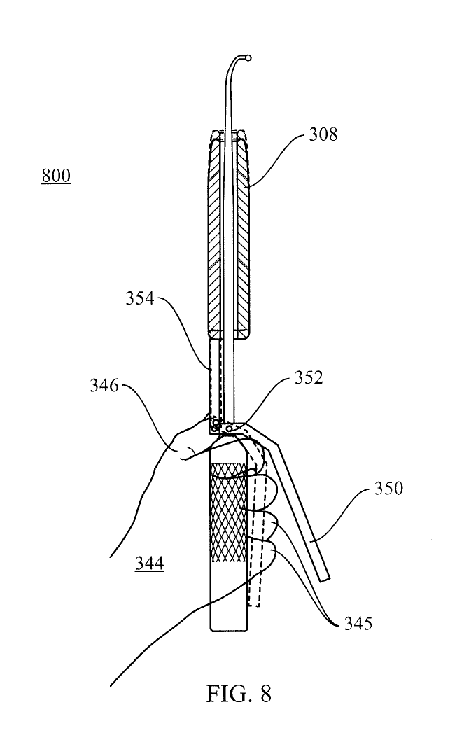

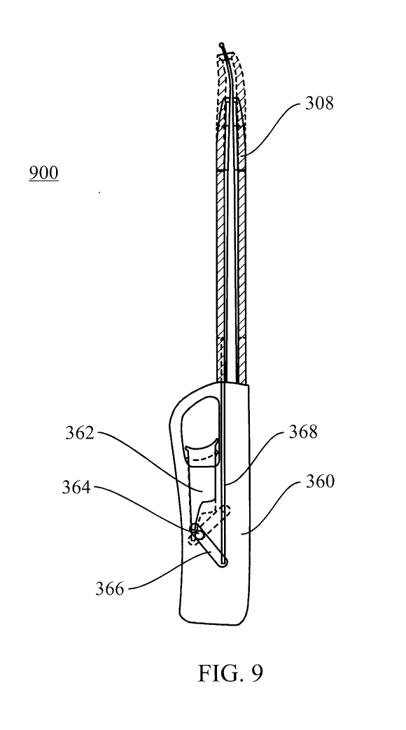

FIG. 8 is a schematic, partial cut-away view of another sinus dilation system 800 in accordance with an illustrative embodiment. The sinus dilation system 800 may include a lever 350, pivots 352, and a driver 354. The lever 350 may be gripped by the fingers 345 of the hand 344 to advance the slidable bougie 308. The lever 350 and the driver 354 may include one or more linkages, pivots, or connectors for moving the driver 354 over a distant significant enough to perform the dilation procedure. According to one aspect, actual displacement of the slidable bougie 308 can be configured as a ratio (e.g. 1/1, 2/1, 4/1 or oppositely 1/2, 1/4) of the total/partial movement of the lever 350. For example, the connection between the lever 350 and the driver 354 may be configured with a cam, whereby the lever 350 rotates the cam about a pivot point and the cam drives the slidable bougie 308 forward. Cammed movement of the slidable bougie 308 could be configured to control the displacement of the slidable bougie 308 at different rates depending upon the position of the lever 350. For example, the initial movement of the lever 350 could be configured to result in a large or small displacement of the slidable bougie 308. The later movement of the lever 350 could also be configured to result in a large or small displacement of the slidable bougie 308. In one aspect, the cammed movement could be configured so the displacement ratio of the slidable bougie 308 near the dilation tip 305 is relatively small compared to the lever 350 displacement.

FIG. 9 is a schematic, partial cut-away view of another sinus dilation system 900 in accordance with an illustrative embodiment. The sinus dilation system 900 may include a handle 360, a trigger 362, a pivot 364, a lever 366, and a driver 368. As shown, the handle 316 may be reconfigured to secure various components of the sinus dilation system 900. In one embodiment, the trigger 362 may be driven by a finger of a medical professional. The trigger 362 may be elongated (e.g. trigger pull length increased) for interacting with the lever 366. The lever 366 may be pivotally attached to the trigger 362, handle 360, and the driver 368. In one embodiment, the motion of the trigger 362 may be transferred to the lever 366 which moves the driver 368 along the longitudinal axis of the sinus dilation system 900 to drive the slidable bougie 308. A lever driving surface of the trigger 362 may be cammed and/or cogged to drive the lever 366 at a desired ratio relative to displacement of the trigger 362.

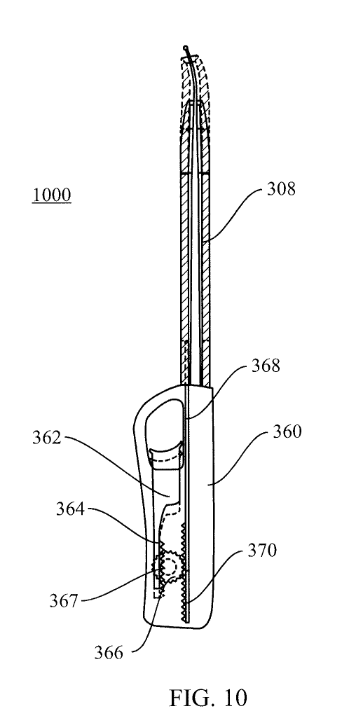

Turning now to FIG. 10 illustrating another bougie dilation system 1000 in accordance with an illustrative embodiment. In one embodiment, the bougie dilation system 1000 may include teeth 364, wheel 366, pivot 367, teeth 370 (e.g. rack), and the driver 368. The trigger 362 may include the teeth 364 for driving the motion of the wheel 366 (e.g. pinion) about the hub 367. For example, the wheel 366 may be rotationally attached to the hub 367. The hub 367 may be attached to one or more portions of the handle 360. The wheel 366 may also include teeth or protrusions for interacting with the teeth 364 and the teeth 370 of the driver 368. The trigger 362, teeth 364 and 370, wheel 366, and driver 368 may interact to drive the motion of the slidable bougie 308. As the trigger 362 is depressed, the teeth 364 drive the wheel 366 which rotates about the hub 367 to drive the teeth 370 and the connected driver 368 thereby advancing the slidable bougie 308. The relative size difference between the diameter of the hub 367 and wheel 366 may be configured to control the ratio of displacement between the trigger 362 and the slidable bougie 308.

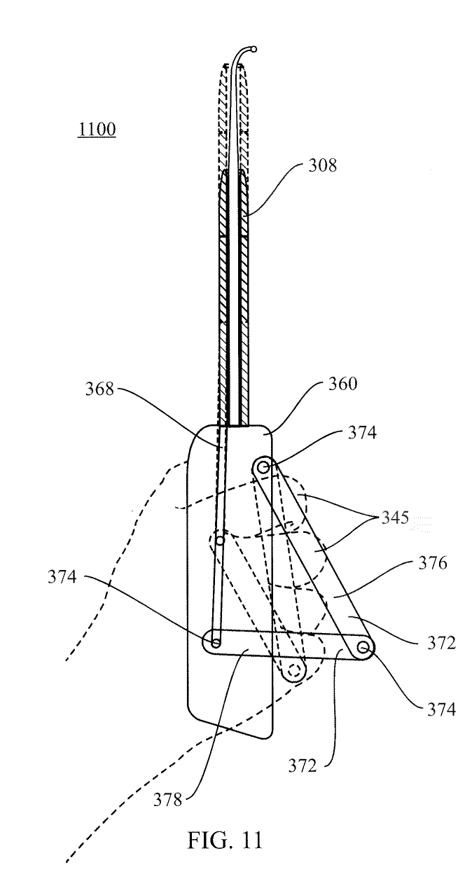

FIG. 11 is a schematic, partial cut-away view of another bougie dilation system 1100 in accordance with an illustrative embodiment. The bougie dilation system 1100 may include a linkage 372, pivots 374, and the driver 368. The linkage 372 as shown includes at least two links. A first end of a first link 376 may be rotationally attached to the handle 360 by one of the pivots 374. A second end of the first link 376 may be rotationally attached to a first end of a second link 378. The second end of the second link 378 may be rotationally attached to the driver 368. In one embodiment, the motion of the linkage 372 and driver 368 may be constrained by one or more channels, guides or cavities defined within the handle 360. The linkage 372 may act as a grip that when squeezed by the fingers 345 moves the driver 368 to advance and retract the slidable bougie 308.

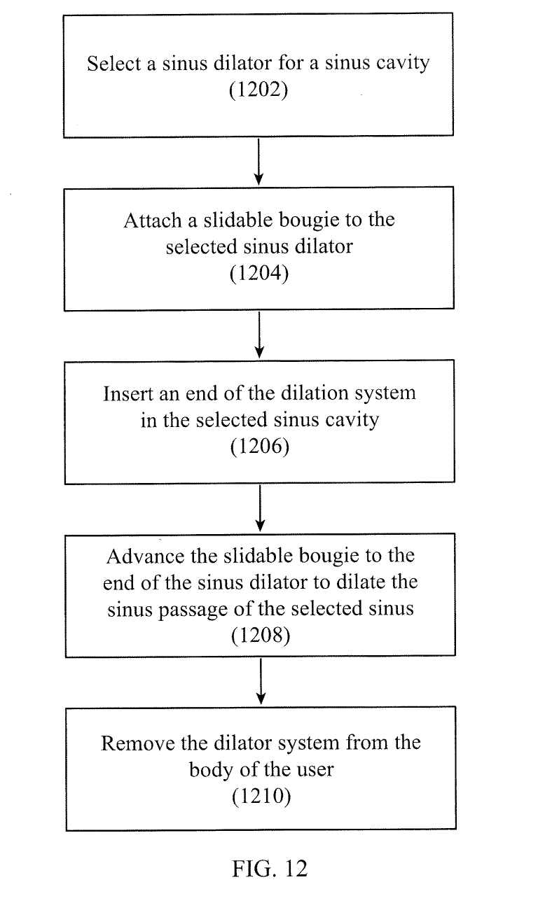

FIG. 12 is a flowchart of a process for utilizing a sinus dilator in accordance with an illustrative embodiment. The process of utilizing a sinus dilator may vary based on the preferences of the medical professional and the circumstances of the user/patient.

In one embodiment, a sinus dilator is selected for a sinus procedure (step 1202). The sinus dilator may be selected from a number of sinus dilators. In one embodiment, the sinus dilator is a sinus seeker, such as those manufactured by Entellus, Invotec, Millennium Surgical Corp., JEDMED, Bausch+Lomb Instruments, and BR Surgical to name a few. The sinus dilator may be singled-sided or multiple-sided. For example, the dilation system utilized with the sinus dilator may be configured to perform sinus dilation of both the frontal and maxillary sinuses.

Next, the medical professional attaches a slidable bougie to the selected sinus dilator (step 1204). The slidable bougie is configured to be advanced and retracted along a length of the sinus dilator. In one embodiment, the slidable bougie may be slipped over the sinus dilator from one or more ends of the sinus dilator. In another embodiment, the slidable bougie may be composed of a single or multiple components, such as a more rigid driving section (moved or pushed by the medical professional) and a more flexible section configured to flex around the end of the sinus dilator to enter the sinus or other region. The distinct components may be attached utilizing threads, ports, connectors, an interference fit, collars, or other mechanical components, features, or structures of the slideable bougie.

The slidable bougie may include an advancement section that is driven by the hand, finger, or thumb of a user or by a mechanical components of the dilation system, such as a driving wheel, lever, or other drive component. The advancement section of the slidable bougie may also require position, securing, or insertion for utilization.

Next, the medical professional inserts an end of the dilation system in the selected sinus cavity (step 1206). The dilator system may also be utilized in any number of other orifices, cavities, or body parts whether naturally or surgically created. In one embodiment, the sinus dilator may be utilized as a seeking tool to find the opening to the sinus and then positioned for performing dilation by advancing the slidable bougie into the sinus pathway.

Next, the medical professional advances the slidable bougie to the end of the sinus dilator to dilate the sinus passage of the selected sinus (step 1208). For example, the sinus dilator may be moved to a desired depth within the sinus of the user and then the medical professional advances the bougie to perform the sinus dilation. In one embodiment, the slidable bougie may not extend beyond the end of the sinus dilator. In another embodiment, the slidable bougie may extend beyond the end of the sinus dilator.

Next, the medical professional removes the dilation system from the body of the user (step 1210). In one embodiment, the dilation system may be broken down to be disposed of, recycled or reused. For example, all or a portion of the bougie may be disposed of and the sinus dilator may be cleaned utilizing accepted medical cleaning equipment, systems, and processes. In another embodiment, the entire dilation system may be disposed of.

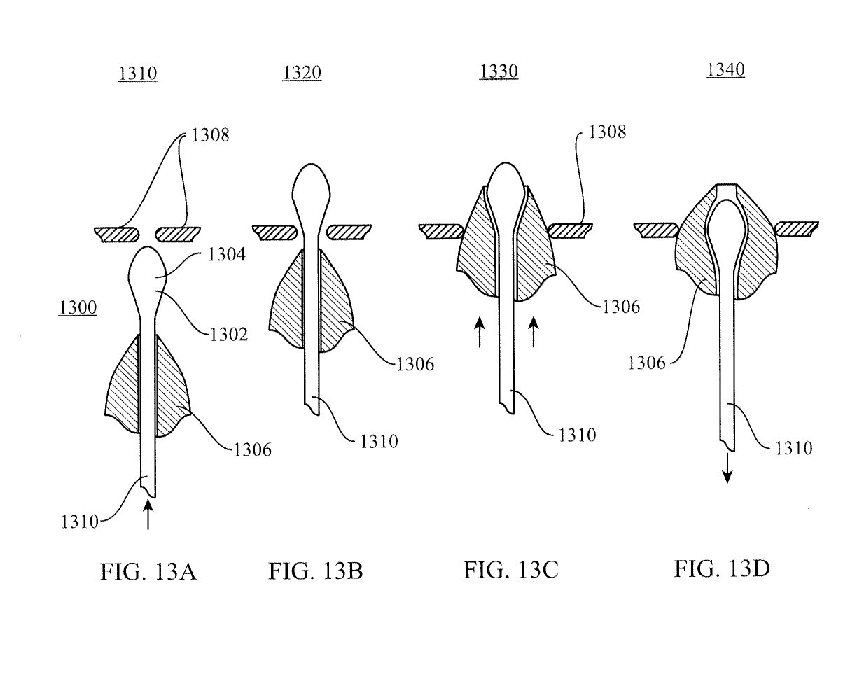

FIG. 13A-D is a schematic, cut-away view of a bougie being utilized for sinus dilation in accordance with an illustrative embodiment. In one embodiment, a dilating portion of a dilation system 1300 may include a sinus dilator 1302, a dilation tip 1304, a slidable bougie 1306, and sinus 1308. The sinus 1308 may represent the sinus cavity being dilated. The dilation system 1300 is show in various phases 1310, 1320, 1330, and 1340 of dilation in FIGS. 13A-13D. In phase 1310, the dilation system 1300 is inserted into the nose of the user and is being utilized to find the sinus 1308. In phase 1320, the dilation tip 1304 of the sinus dilator 1302 is inserted into the sinus 1308. Many medical professionals are already accustomized to utilizing a sinus dilator, such as a sinus seeker to find the location of the user's sinuses. During phase 1330, the slidable bougie 1306 is advanced into the sinus 1308 in accordance with one or more of the herein-described techniques. In one embodiment, the dilation tip 1304 may have a greater diameter than a shaft 1310 of the sinus dilator 1302. As a result, in phase 1330 and 1340, the slidable bougie 1306 may further expand the sinus 1308 when moving over the dilation tip 1304 because of the increased diameter.

In one embodiment, the slidable bougie 1306 is advanced and retracted over the sinus dilator 1302. In another embodiment, the slidable bougie 1306 may be held still while the sinus dilator is advanced and retracted to dilate the sinus 1308.

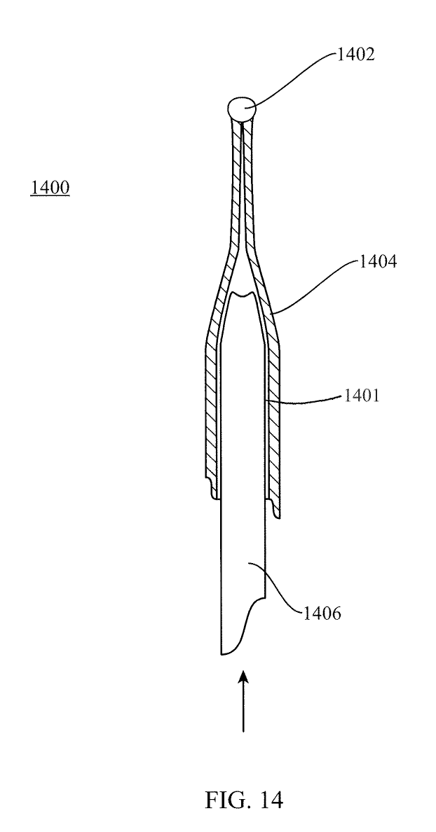

FIG. 14 is a schematic, partial cut-away of another dilation system 1400 in accordance with an illustrative embodiment. The dilation system 1400 may include a flexible nasal dilator 1401 that may include a dilation tip 1402 and flexible walls 1404. The flexible walls 1404 are connected to the dilation tip 1402 and constructed of a sufficiently rigid material component whereby in the unseparated configuration (e.g. shown above the bougie) the dilation tip 1402 is capable of navigating, probing for and identifying a patient's sinuses. The dilation system 1400 may be configured to receive the bougie 1406 within the flexible walls 1404 at a separate end (not shown). The flexible walls 1404 may be configured to expand in response to the bougie 1406 being advanced all of the way to the dilation tip 1402. The material component may include a formable skeleton encased by a medical grade skin material, a flexible constituent (e.g. rubber, plastic, thin metal/alloy), or a shape memory material.

The dilation system 1400 may be configured to advance the bougie 1406 utilizing any number of systems, components, and methods that are herein described. For example, the medical professional may advance the bougie 1406 using a thumb, finger trigger, drive wheel, lever, cam, or so forth.

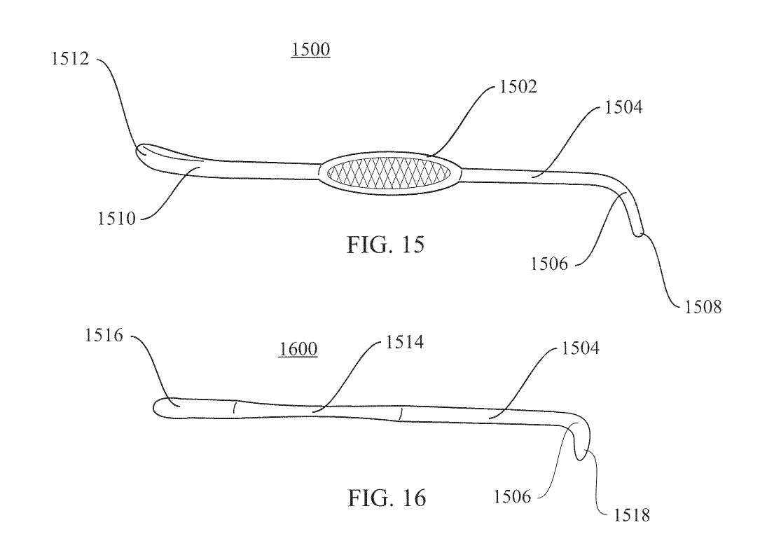

FIG. 15 is a schematic, plan views of a sinus dilator 1500 in accordance with an illustrative embodiment. The sinus dilator 1500 is configured to be utilized in one or more distinct sinuses. In one embodiment, the sinus dilator 1500 may include duplicate or different dilation heads disposed at each end for treating both of the frontal, sphenoid, or maxillary sinuses. The sinus dilator may include a grip 1502, body 1504, a neck 1506, a dilation tip 1508, a neck 1510, and a dilation tip 1512. The sinus dilator 1500 may utilize any of the configurations shown in FIGS. 1A-1D, and 2A-2E or the other described embodiments. The grip 1502 may be rounded or flattened based on the preferences of the medical professional. The grip 1502 may also include knurling, ribbing, corrugations, undulations, extensions, rubber, or other highly tactile and easily gripped surfaces. For example, the dilation tip 1508 may be utilized to access the maxillary sinus(es) and the dilation tip 1512 may be utilized to access the sphenoid sinus(es). The body 1504 of the sinus dilator 1500 may be configured with attachment points (e.g. similar to the release 136 shown in FIG. 1D) to removably attach different dilation heads to either end of the body 1504.

FIG. 16 is a schematic, plan views of a sinus dilator 1600 in accordance with an illustrative embodiment. The sinus dilator 1600 may additionally include a grip 1514 and dilation tip 1516.

In one embodiment, a medical professional may have access to a number of sinus dilators for the distinct sinuses and of distinct sizes as well. For example, the dilation tips may vary in configuration as well as the overall diameter utilized for performing dilation that may vary between 1 mm-6 mm. However, other larger and smaller sizes may be available for infants, and individuals that require larger sizes.

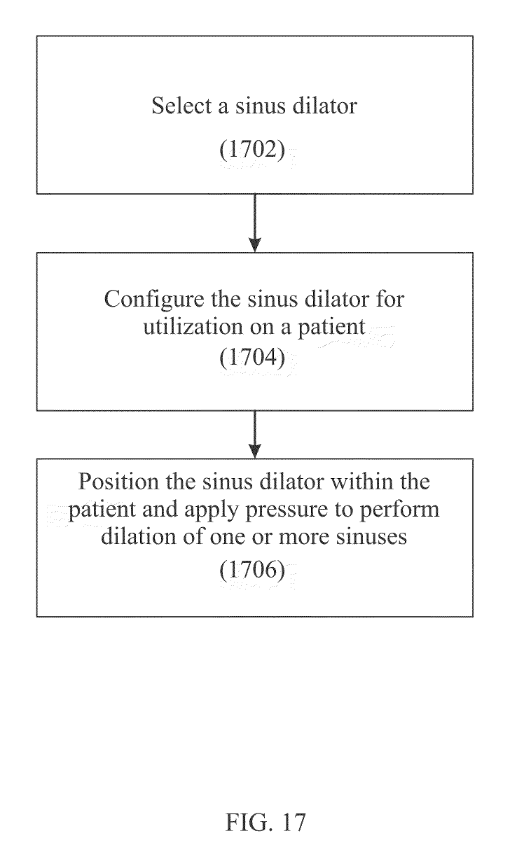

FIG. 17 is a flowchart of a process for utilizing a sinus dilator in accordance with an illustrative embodiment. The process of utilizing a sinus dilator may vary based on the preferences of the medical professional and the circumstances of the user/patient. Although described for human sinuses, the dilation process and procedures herein described may be applied to any number of dilation procedures for humans, animals, or mechanical components.

In one embodiment, a sinus dilator is selected for a sinus procedure (step 1202). The sinus dilator may be selected from a number of sinus dilators varying from 1 mm-6 mm in diameter at the widest for the dilating portion of the dilation head. For example, the dilation needs of adults, children, or individual users may vary based on the configuration of their body. In addition, distinct types of dilation tips may be utilized. The sinus dilator may be singled-sided or multiple-sided. For example, the sinus dilator may be configured to perform sinus dilation of both the frontal and maxillary sinuses.

Next, the sinus dilator is configured for utilization on a user (step 1704). In one embodiment, the medical professional may bend, manipulate, or configure one or more portions or sections of the sinus dilator to best fit the patient for dilation. For example, the sinus dilator may be configured for reconfiguration to allow for custom utilization. During step 1704, one or more dilation heads or ends may be connected to a handle or base portion of the sinus dilator. For example, dilation heads for performing dilation of the frontal and maxillary sinuses may be attached to the sinus dilator (e.g. snapped into position, screwed in, etc.).

Next, the sinus dilator is positioned within the user and pressure is applied to perform dilation of one or more sinuses (step 1706). The medical professional may careful determine the location of the sinus utilizing the dilation tip of the sinus dilator before performing the dilation process. The medical professional may utilize any number of three dimensional forces determined to be necessary to dilate the tissue, bone, membranes, and other portions of the sinuses to perform dilation that will best help the patient.

The previous detailed description is of a small number of embodiments for implementing the invention and is not intended to be limiting in scope. The following claims set forth a number of the embodiments of the invention disclosed with greater particularity.

What is claimed:

1. A nasal dilator, comprising:
an elongated member terminating in an end;
a slidable member having a terminal end movable to the end of the elongated member in a first position; and
a dilation portion at the end of the elongated member, the dilation portion having a first diameter and a second diameter greater than the first diameter when the slidable member is in the first position.

2. The nasal dilator of claim 1, wherein the slidable member encircles at least a portion of the elongated member in the first position for performing dilation.

3. The nasal dilator of claim 1, wherein the slidable member comprises one or more sections that are positioned over the elongated member in a second position opposite the first position, wherein the one or more sections are advanced along the elongated member to the first position to perform dilation.

4. The nasal dilator of claim 1, wherein the slidable member includes one or more grips for advancing the slidable member utilizing one or more fingers or thumbs of a medical professional.

5. The nasal dilator of claim 1, wherein the slidable member includes a thumb advancement extending from one or more sections of the slidable member.

6. The nasal dilator of claim 1 further comprising:
a drive wheel for advancing the slidable member along the elongated member.

7. The nasal dilator of claim 1, wherein the end of the elongated member comprising a sphenoid dilation tip, frontal dilation tip, or maxillary dilation tip.

8. The nasal dilator of claim 1, wherein the end of the elongated member is replaceable with a number of ends including curvatures adapted for a particular sinus.

9. The nasal dilator of claim 1, wherein the end of the elongated member includes a sinus seeker that facilitates a medical professional in finding a sinus through exploration.

10. The nasal dilator of claim 1, wherein an inside surface of the slidable member is low-friction to facilitate sliding of the slidable member along the elongated member.

11. A nasal dilator with bougie, comprising:
a shaft terminating in a tip;
a slidable bougie having a dilation end movable from a retracted position to at least the tip of the shaft in a dilation position; and
the nasal dilator having a first diameter at the tip in the retracted position and a second diameter at the tip greater than the first diameter in the dilation position.

12. The nasal dilator of claim 11, wherein the slidable bougie expands the tip from the first to the second diameter during movement from the retracted to the dilation position.

13. The nasal dilator of claim 11 further comprising:
an actuator in operable connection with the slidable bougie, the actuator having an actuated position corresponding with the dilation position of the slidable bougie.

14. The nasal dilator of claim 11, wherein at least a portion of the shaft is housed in the slidable bougie.

15. The nasal dilator of claim 11, wherein the tip of the shaft is housed in the slidable bougie in the dilation position.

16. A nasal dilation method, comprising:
providing an elongated member terminating in an end and a slidable member having a terminal end;
moving the slidable member to a first position at the end of the elongated member; and
changing a dilation portion at the end of the elongated member from a first diameter to a second diameter greater than the first diameter by moving the slidable member to the first position.

17. The method of claim 16 further comprising:
expanding the end of the elongated member to the second diameter.

18. The method of claim 16 further comprising:
moving an actuator to an actuated position for moving the slidable member to the first position.

19. The method of claim 16 further comprising:
removing or adding slidable sections to the slidable member.

20. The method of claim 16 further comprising:
dilating a sinus by advancing the slidable member to the first position.
